(19) United States
(12) Patent Application Publication
(10) Pub. No.: US 2007/0219253 A1
(43) Pub. Date: Sep. 20, 2007

(54) Decongestant / antihistaminic / expectorant compositions

(76) Inventors: Gul Balwani, Princeton Junction, NJ (US); Alexander D. D'Addio, Piscataway, NJ (US); Jeff Gazzara, Mount Laurel, NJ (US); Phuong Grace Dang, West Windsor, NJ (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/665,220

(22) Filed: Sep. 17, 2003

(51) Int. Cl. A61K 31/44

(52) U.S. Cl. 514/352

(57) ABSTRACT

Compositions consisting essentially of phenylephrine tannate, pyrilamine tannate, and guaifenesin which are effective when administered orally for the symptomatic relief of cough and nasal congestion associated with respiratory tract conditions such as the common cold, bronchial asthma, acute and chronic bronchitis are disclosed.

### FIELD OF INVENTION

The invention relates to a novel decongestant/antihistaminic/expectorant composition containing: phenylephrine tannate, pyrilamine tannate, and guaifenesin.

### BACKGROUND OF THE INVENTION

A considerable number of tannic acids occur in nature. Chemically, these acids are described as polymers of different hydroxybenzoic acids. Generally, when the term tannic acid is employed, as in the present case, the acid referred to is gallotannic acid: the internal ester of gallic acid also frequently referred to as tannin.

Tannic acid, a gallotannin, appears as an amorphous powder or glistening scales, or as spongy masses varying in color from yellowish-white to light brown. Tannic acid is very soluble in water, in alcohol, and in glycerol.

Tannic acids are usually obtained from glycosides which consist of several molecules of a tannic acid in combination with glucose.

Commercially available, tannic acid, also known as tannin, has a complex non-uniform chemistry, usually contains from about 5% to about 10% water by weight, has a molecular weight of about 1700, and is typically produced from Turkish or Chinese nutgall.

Phenylepherine, known chemically as (−)1-m-hydroxy-α-[(methylamino)methyl]benzyl alcohol, is a synthetic, optically active sympathomimetic amine. It is a white, odorless, non-hygroscopic, crystalline compound possessing a bitter taste. It has a melting point of 169 to 172° C. The frequently used hydrochloride salt has a melting point of 140 to 145° C. and is freely soluble in water and in alcohol. It is a directly acting sympathomimetic with strong α-agonist and negligible β-agonist and central nervous activity. It is used as a nasal decongestant.

Pyrilamine is one of the oldest and most enduring antihistaminic drugs, known chemically as N[(4-methoxyphenyl)methyl]-N′,N′-dimethyl-N-2-pyridinyl-1,2-ethanediamine. It is an oily liquid, and its preparation is disclosed in U.S. Pat. No. 2,502,151. Pyrilamine hydrochloride salt is very soluble in water and has a melting point of 143-143.5° C. The maleate salt is soluble in water and in alcohol, and it is slightly soluble in benzene and in ether; it has a melting point of 100-101° C.

Decongestant and antihistaminic compounds in the form of their free bases as well as their salts, e.g. hydrochloride, citrate, maleate, tannate, etc., are well known. Decongestants in the form of their tannate salts are very desirable because such salts are generally stable. Tannate salts are also desirable because they provide a prolonged release of the active ingredient free bases.

Tannate salts are typically prepared by reacting the free base, e.g. phenylephrine, pyrilamine etc. with tannic acid in the presence of a volatile solvent, usually isopropanol. Typically, in the conventional isopropanol route, the free base and the tannic acid will be present in the isopropanol at a concentration of about 20% based on the weight of the reaction mixture. The reaction mixture is stirred for about one hour while maintaining the mixture at 60-70° C. The reaction mixture is cooled to room temperature and then filtered, washed with isopropanol, and then vacuum dried. Alternative routes to the tannate salts are described in U.S. Pat. No. 5,599,846 and U.S. Pat. No. 5,663,415, the disclosures of each of which are hereby incorporated by reference in their entireties.

Guaifenesin, known chemically as 3-(2-methoxyphenoxy)-1,2-propanediol, is a crystalline powder soluble in water and alcohol. It is readily absorbed from the gastrointestinal tract. It is indicated in the 23^{rd} edition of USP Drug Information as an expectorant for the symptomatic relief of cough due to colds and minor upper respiratory infections.

### THE INVENTION

Research and development has shown that a unit dose for oral administration containing the novel combination of phenylephrine tannate, pyrilamine tannate, and guaifenesin can be produced. Phenylephrine tannate provides the nasal decongestion action. Guaifenesin has an expectorant action, which increases the output of respiratory tract fluid by reducing adhesiveness and surface tension. The increased flow of less viscous secretions promotes ciliary action and facilitates the removal of mucus. This changes a dry, unproductive cough to one that is more productive and less frequent. Pyrilamine tannate is an antihistaminic agent with a low incidence of sedative effects. It provides the desired relief from allergic rhinitis symptoms.

The compositions described herein are preferably designed to be taken twice a day with guaifenesin providing its expectorant action, phenylephrine tannate providing a prolonged nasal decongestant action, and pyrilamine tannate providing a prolonged antihistaminic action. The compositions of the present invention may be prepared for oral administration in the form of powders, capsules, elixirs, syrups, and in the preferred forms of tablets and suspensions.

Tablets containing the unique composition of phenylephrine tannate, pyrilamine tannate, and guaifenesin compositions of the present invention are prepared in a conventional manner by the addition of suitable pharmaceutical carriers including fillers, stabilizers, or antioxidants like ascorbic acid and sodium metabisulfite, diluents, colorants, lubricants and the like, as well as conventional and well known binding and disintegrating agents. In a preferred embodiment tablets would contain about 20 to 30 mg of phenylephrine tannate, about 40 to 80 mg pyrilamine tannate, and about 100 to 400 mg of guaifenesin per tablet. The tablet composition of the present invention containing dibasic calcium phosphate, microcrystalline cellulose, methylcellulose, polygalacturonic acid, talc, colorants, colloidal silicon dioxide and magnesium stearate, as described in Example 1 which follows, is illustrative of a tablet formulation of the present invention prepared by well known conventional tableting techniques such as those disclosed in U.S. Pat. Nos. 3,018,221; 2,798,024 and 2,757,124, the disclosures of each of which are hereby incorporated by reference in their entireties. In a particularly preferred embodiment the tablets contain about 25 mg of phenylephrine tannate, about 60 mg of pyrilamine tannate, and about 200 mg of guaifenesin. In another particularly preferred embodiment the tablets contain about 25 mg of phenylephrine tannate, about 60 mg of pyrilamine tannate, and about 300 mg of guaifenesin.

### EXAMPLE 1

Phenylephrine Tannate, Pyrilamine Tannate, and Guaifenesin Tablets

| Ingredient | Milligrams per Tablet |
|---|---|
| Phenylephrine Tannate | 25.00 |
| Pyrilamine Tannate | 60.00 |
| Guaifenesin | 200.00 |
| Calcium Phosphate Dibasic Dihydrate (DiTab ®) | 96.10 |
| Microcrystalline Cellulose (ProSolv ®) | 195.00 |
| Methylcellulose, 1500, USP | 39.00 |
| Polygalacturonic Acid | 13.00 |
| Talc, USP | 12.00 |
| FD&C Blue #1 Lake 29% | 5.00 |
| FD&C Red #40 Lake 40% | 1.00 |
| Colloidal Silicone Dioxide, NF | 2.60 |
| Magnesium Stearate, NF | 1.30 |

Suspensions containing the unique composition of phenylephrine tannate, pyrilamine tannate and guaifenesin of the present invention are prepared in a conventional manner. In a preferred embodiment the suspensions of the present invention contain about 3 to 15 mg of phenylephrine tannate, about 25 to 35 mg pyrilamine tannate, and about 50 to 300 mg of guaifenesin, per 5 ml of suspension (one teaspoon). Additionally, the suspension formulations may contain colorants; natural and artificial flavors; glycerin; kaolin; pectin; magnesium aluminum silicate; methylparaben; benzoic acid; purified water; stabilizers like ascorbic acid and sodium metabisulfite; and sweeteners like saccharin, sucralose, and sucrose. Example 2, which follows, is illustrative of a suspension formulation of the present invention prepared by conventional well known compounding techniques. In a particularly preferred embodiment the suspensions contain about 5 mg of phenylephrine tannate, about 30 mg of pyrilamine tannate, and about 100 mg of guaifenesin, per 5 ml of suspension (one teaspoon). In another particularly preferred embodiment the suspensions contain about 5 mg of phenylephrine tannate, about 30 mg of pyrilamine tannate, and about 200 mg of guaifenesin, per 5 ml of suspension (one teaspoon).

### EXAMPLE 2

Phenylepherine Tannate, Pyrilamine Tannate, and Guaifenesin Suspension

| Ingredient | Milligrams per 5 ml. |
|---|---|
| Phenylephrine tannate | 5.00 |
| Pyrilamine tannate | 30.00 |
| Guaifenesin | 100.00 |
| Pectin, USP (Medium Viscosity) | 57.00 |
| Kaolin, USP (Colloidal Powder) | 680.00 |
| Magnesium Aluminum Silicate, NF | 35.00 |
| Benzoic Acid, USP | 10.00 |
| Methylparaben, NF | 2.50 |
| Sucrose, NF | 1818.00 |
| Saccharin Sodium, USP | 3.50 |
| Glycerin, USP | 915.00 |
| Flavor Grape | 9.00 |
| Dye Purple Shade R | 0.24 |
| FD&C Blue #1 Dye | 0.48 |
| Purified or Deionized Water, USP adjust to | 5 mL |

Sodium Hydroxide, Tannic Acid, Sodium Citrate, and Citric Acid may also be included in the formula for pH adjustment.

For the purpose of this disclosure, a warm-blooded animal is a member of the animal kingdom possessed of a homeostatic mechanism and includes mammals and birds. The warm-blooded animal that is preferably treated is a human.

The dosage administered will be dependent on the age, health, and weight of the recipient, kinds of concurrent treatment, if any, frequency of treatment and effect desired.

In general, the named three active components are the only active components used in the composition.

It should be understood that the foregoing disclosure and examples will enable one of ordinary skill in the art to practice the best mode of the invention. However, it is anticipated that numerous variations will occur to those skilled in the art. A latitude of modification, substitution, and change is intended and in some instances, some features of the invention will be employed without a corresponding use of other features. Accordingly, it is intended that the spirit and scope of the invention disclosed herein should be limited only by the following claims.

What is claimed is:

1. A therapeutic composition for the symptomatic relief of cough and nasal congestion associated with adverse respiratory tract conditions in warm-blooded animals in need of such treatment, said composition comprising pharmaceutically effective amounts of active ingredients, wherein said active ingredients consist of phenylephrine tannate, pyrilamine tannate, and guaifenesin.

2. The therapeutic composition of claim 1, in tablet form.

3. The therapeutic composition of claim 2, wherein each tablet contains about 20 to 30 mg of phenylephrine tannate, about 40 to 80 mg of pyrilamine tannate, and about 100 to 400 mg of guaifenesin.

4. The therapeutic composition of claim 2, wherein said tablet form contains about 25 mg of phenylephrine tannate, about 60 mg of pyrilamine tannate, and about 200 mg of guaifenesin.

5. The therapeutic composition of claim 2, wherein said tablet form contains about 25 mg of phenylephrine tannate, about 60 mg of pyrilamine tannate, and about 300 mg of guaifenesin.

6. The therapeutic composition of claim 1, in suspension form.

7. The therapeutic composition of claim 6, wherein said suspension form contains about 3 to 15 mg of phenylephrine tannate, about 25 to 35 mg of pyrilamine tannate, and about 50 to 300 mg of guaifenesin, per 5 ml of suspension.

8. The therapeutic composition of claim 6, wherein said suspension form contains about 5 mg of phenylephrine tannate, about 30 mg of pyrilamine tannate, and about 100 mg of guaifenesin, per 5 ml of suspension.

9. The therapeutic composition of claim 6, wherein said suspension form contains about 5 mg of phenylephrine tannate, about 30 mg of pyrilamine tannate, and about 200 mg of guaifenesin, per 5 ml of suspension.

10. A method for symptomatically treating and relieving the distress of cough and nasal congestion associated with adverse respiratory tract conditions in warm-blooded animals, comprising orally administering to warm-blooded animals in need of such treatment the composition of claim 1.

11. The method of claim 10, wherein said composition is in tablet form.

12. The method of claim 11, wherein each tablet contains about 20 to 30 mg of phenylephrine tannate, about 40 to 80 mg of pyrilamine tannate, and about 100 to 400 mg of guaifenesin.

13. The method of claim 11, wherein said tablet form contains about 25 mg of phenylephrine tannate, about 60 mg of pyrilamine tannate, and about 200 mg of guaifenesin.

14. The method of claim 11, wherein said tablet form contains about 25 mg of phenylephrine tannate, about 60 mg of pyrilamine tannate, and about 300 mg of guaifenesin.

15. The method of claim 10, wherein said composition is in suspension form.

16. The method of claim 15, wherein said suspension form contains about 3 to 15 mg of phenylephrine tannate, about 25 to 35 mg of pyrilamine tannate, and about 50 to 300 mg of guaifenesin, per 5 ml of suspension.

17. The method of claim 15, wherein said suspension contains about 5 mg of phenylephrine tannate, about 30 mg of pyrilamine tannate, and about 100 mg of guaifenesin, per 5 ml of suspension.

18. The method of claim 15, wherein said suspension contains about 5 mg of phenylephrine tannate, about 30 mg of pyrilamine tannate, and about 200 mg of guaifenesin, per 5 ml of suspension.

19. The method of claim 10, wherein said oral administration is a twice a day administration.
